(19) United States
(12) Patent Application Publication
(10) Pub. No.: US 2022/0331210 A1
(43) Pub. Date: Oct. 20, 2022

(54) ACRYLATE AND SILICONE-FREE COSMETIC O/W EMULSION

(71) Applicant: BEIERSDORF AG, Hamburg (DE)

(72) Inventors: Kerstin FRANCK, Quickborn (DE); Kerstin SKUBSCH, Prisdorf (DE)

(21) Appl. No.: 17/753,344

(22) PCT Filed: Jul. 27, 2020
(86) PCT No.: PCT/EP2020/071104

§ 371 Date: Feb. 28, 2022

(30) Foreign Application Priority Data
Aug. 28, 2019 (DE) 10 2019 212 891.2

(51) Int. Cl. A61K 8/06; A61K 8/36; A61K 8/73; A61Q 19/00

(52) U.S. Cl. CPC A61K 8/062; A61K 8/361; A61K 8/732; A61Q 19/00; A61K 2800/34

(57) ABSTRACT

The invention relates to a cosmetic emulsion.

In general, cosmetic products are not only used for looking beautiful and attractive, but their effect makes a decisive contribution to increased self-esteem and the well-being of people. Accordingly, a wide variety of cosmetic products are used for daily cleansing and care of human skin.

Skin care products usually consist of emulsions. Emulsions are generally understood to mean heterogeneous systems consisting of two liquids that are immiscible or miscible only to a limited extent with one another, which are typically referred to as phases and in which one of the two liquids is dispersed in the other liquid in the form of very fine droplets. Externally and viewed with the naked eye, emulsions appear homogeneous.

If the two liquids are water and oil and oil droplets are present finely distributed in water, then it is an oil-in-water emulsion (O/W emulsion, for example milk). The basic character of an O/W emulsion is characterized by the water. With a water-in-oil emulsion (W/O emulsion, for example butter), the reverse principle is the case, in which the basic character here is determined by the oil.

To stabilize and thicken O/W emulsions, acrylate-based polymers are usually incorporated into these formulations. Acrylate-based polymers are polymers obtained from homo- or copolymerization with acrylic acid and/or methacrylic acid. Examples of these are, inter alia, carbomer or acrylate copolymer.

The prior art includes, inter alia, the documents DE 10148825 A1, DE 19938756 A1 and DE 29924371 U1, which disclose O/W care emulsions with acrylate-based polymers. However, the use of these acrylate-based polymers is increasingly being criticized since the biodegradability thereof has not been fully clarified.

Another frequently used constituent of O/W emulsions for application to the skin are silicone oils. When an O/W emulsion is applied, the skin feels supple. In addition, silicone oils have a softening effect and act as an optical wrinkle filler. Silicones are not harmful to the skin per se. As substances foreign to the skin, they do not trigger any allergies or irritation reactions. Once washed off, the skin is how it was before.

However, the fact that the environmental compatibility of silicone oils has not yet been conclusively assessed and that the substances are degraded in nature only with difficulty is also critical. Consequently, it is necessary to reduce the use of silicone oils in cosmetic preparations for application to the skin.

The problem, however, is the fact that O/W emulsions that do not comprise any acrylate-containing polymers and in which silicone oils are not used, often tend to leave white residues when spread on the skin.

The object of the present invention was therefore to provide an acrylate- and silicone-free cosmetic O/W emulsion which does not have the disadvantages of the prior art. In particular, it was an object to provide such an O/W emulsion which is quickly absorbed when applied and which has lower amounts of residues.

Surprisingly, the object(s) is/are achieved by an acrylate- and silicone-free cosmetic O/W emulsion containing
a) from 0.1 to 6% by weight of at least one fatty acid having 12 to 22 carbon atoms, and
b) from 0.1 to 5% by weight hydroxypropyl starch phosphate;

based in each case on the total weight of the emulsion.

The invention also relates to the use of a mixture of
a) at least one fatty acid having 12 to 22 carbon atoms, and
b) hydroxypropyl starch phosphate,
in an acrylate- and silicone-free cosmetic O/W emulsion for reducing white residues when the emulsion is applied to the skin.

If weight percentages (% by weight) are given below without reference to a specific composition or specific mixture, these data always relate to the total weight of the cosmetic O/W emulsion. If ratios of components/substances/groups of substances are disclosed below, these ratios relate to weight ratios of the specified components/substances/groups of substances.

If weight percentage ranges are given below for the constituents of the cosmetic O/W emulsion, the disclosure of the present application also includes all individual values in steps of 0.1% by weight within these weight percentage ranges.

In the context of the present disclosure, the phrases “according to the invention”, “advantageous according to the invention”, “advantageous in the context of the present invention” etc. always refer to both the preparation according to the invention and to the use according to the invention.

Unless otherwise stated, all tests were carried out under standard conditions. The term “standard conditions” means 20° C., 1013 hPa and a relative humidity of 50%.

If the term skin is used, this preferably refers to human skin.

The O/W emulsion according to the invention is acrylate-free. In the context of the present invention, acrylate-free means that the total fraction of acrylate-based polymers is less than 0.05% by weight and particularly preferably is 0% by weight, the figures being based on the total weight of the emulsion. According to the invention, acrylate-based polymers are understood to mean all polymers which are obtained from a homo- or copolymerization with acrylic and/or methacrylic acid.

In the context of the present disclosure, silicone-free means that no molecules containing dialkylsiloxane units are present.

According to the invention, the cosmetic O/W emulsion comprises at least one fatty acid having 12 to 22 carbon atoms. Preferred fatty acids are selected from the group palmitic acid, stearic acid, myristic acid, arachidic acid and oleic acid.

It is preferred if at least palmitic acid and/or stearic acid are present.

The total fraction of fatty acids having 12 to 22 carbon atoms in the emulsion according to the invention is from 0.1 to 6% by weight, based on the total weight of the emulsion. It is preferred if the total fraction of fatty acids having 12 to 22 carbon atoms is from 0.2 to 5% by weight and particularly preferably from 0.5 to 3% by weight, based in each case on the total weight of the emulsion.

It is also advantageous if at least palmitic acid is present and the fraction of palmitic acid is preferably from 0.4 to 2.2% by weight, based on the total weight of the emulsion.

It is also advantageous if at least stearic acid is present and the fraction of stearic acid is preferably from 0.4 to 2.2% by weight, based on the total weight of the emulsion.

Advantageous embodiments of the invention are further characterized in that, in addition to palmitic acid and stearic acid, other fatty acids are present, the ratio by weight of the total amount of palmitic acid and stearic acid to the total amount of other fatty acids being from 8:1 to 99:1.

Furthermore, it is advantageous in the context of the present invention if the fraction of hydroxypropyl starch phosphate is from 0.2% by weight to 3.8% by weight, preferably from 0.3% by weight to 3.5% by weight and particularly preferably from 0.4% by weight to 3% by weight, based on the total weight of the emulsion.

In general, hydroxypropyl starch phosphate is an esterification product based on starch. According to the invention, it is possible to use hydroxypropyl starch phosphate based on different starches. Those skilled in the art are familiar with, inter alia, wheat or potato starch.

Advantageous embodiments of the present invention are characterized in that the hydroxypropyl starch phosphate used is an esterification product based on corn starch. Within these embodiments, it is further preferred if the fraction of the hydroxypropyl starch phosphate, which is an esterification product based on corn starch, is from 0.2% by weight to 3.8% by weight, preferably from 0.3% by weight to 3.5% by weight and particularly preferably from 0.4% to 3% by weight, based on the total weight of the emulsion.

A hydroxypropyl starch phosphate according to the invention based on corn starch can be obtained under the trade name C*HiForm A 12747 from Cargill or Structure® XL from Akzo Nobel Specialty Chemicals.

It is also advantageous according to the invention if glyceryl stearate is present. If glyceryl stearate is present, it is also advantageous if the total fraction of glyceryl stearate is from 0.1% by weight to 5.0% by weight, preferably 0.5% by weight to 3.2% by weight and particularly preferably from 1.0% by weight to 2.5% by weight, the figures being based on the total weight of the emulsion.

It is also advantageous if glyceryl stearate citrate is present. If glyceryl stearate citrate is present, it is also advantageous if the total fraction of glyceryl stearate citrate is from 0.1% by weight to 5.0% by weight, preferably 0.5% by weight to 3.2% by weight and particularly preferably from 0.8% by weight to 2.5% by weight, the figures being based on the total weight of the emulsion.

It is also advantageous if polyglyceryl-3 diisostearate is present. If polyglyceryl-3 diisostearate is present, it is also advantageous if the total fraction of polyglyceryl-3 diisostearate is from 0.1% by weight to 3.1% by weight, preferably 0.15% by weight to 1.5% by weight and particularly preferably from 0.2% by weight to 1.0% by weight, the figures being based on the total weight of the emulsion.

It is also advantageous if polyglyceryl-2 caprate is present. If polyglyceryl-2 caprate is present, it is also advantageous if the total fraction of polyglyceryl-2 caprate is from 0.1% by weight to 0.5% by weight, preferably 0.15% by weight to 0.4% by weight, the figures being based on the total weight of the emulsion.

Furthermore, it was surprisingly found that emulsions which in particular can be stabilized by the combination according to the invention are characterized in that the fraction of the oil phase of the emulsion is from more than 2% by weight to 30% by weight, preferably from more than 5% by weight to 20% by weight and particularly preferably from more than 8% by weight to 15.5% by weight, based on the total weight of the emulsion, with surfactants and emulsifiers by definition not being included in the oil phase, unless they are explicitly assigned to the oil phase in the present disclosure. Surprisingly, emulsions having a large proportion of oil phases can thus be stabilized.

According to the invention, emulsifiers and surfactants are not included in the oil phase. This means that glyceryl stearate, glyceryl stearate citrate and polyglyceryl-3 diisostearate, inter alia, are not included in the oil phase.

Emulsifiers are understood to mean all substances that are listed in the International Cosmetic Ingredient Dictionary and Handbook, Thirteenth Edition 2010, (ISBN 1-882621-47-6) under the designation “emulsifying agent”. Surfactants are understood to mean all substances that are listed in the International Cosmetic Ingredient Dictionary and Handbook, Thirteenth Edition 2010, (ISBN 1-882621-47-6) under the designation “surfactant”.

Surprisingly, it has also been shown that the consistency and thus the viscosity of the O/W emulsion according to the invention could be further increased by adding one or more fatty alcohols having 14 to 22 carbon atoms to the emulsion. The total fraction of these fatty alcohols having 14 to 22 carbon atoms is advantageously from 0.1% by weight to 8.0% by weight, preferably 0.5% by weight to 7.0% by weight and particularly preferably from 1% by weight to 6.0% by weight, based on the total weight of the emulsion. Fatty alcohols having 14 to 22 carbon atoms are included in the oil phase.

Fatty alcohols to be selected with particular preference are myristyl alcohol, cetyl alcohol and/or stearyl alcohol, these preferably being used at a total fraction of 0.1% by weight to 8.0% by weight, preferably 2% by weight to 7.0% by weight and particularly preferably from 3% by weight to 6.0% by weight, based on the total weight of the emulsion. The mixture of cetyl alcohol and stearyl alcohol is known by the INCI name cetearyl alcohol.

Advantageous embodiments of the present invention are further characterized in that the O/W emulsion comprises further polysaccharide-containing polymers.

Other advantageous embodiments of the present invention are further characterized in that the emulsion does not comprise any other starches and starch derivatives apart from hydroxypropyl starch phosphate. It was particularly surprising that it was possible to dispense with the additional use of further polymers.

Other advantageous embodiments of the present invention are further characterized in that the emulsion comprises no celluloses or cellulose derivatives.

Other advantageous embodiments of the present invention are further characterized in that the emulsion comprises no other polysaccharide-containing polymers.

The O/W emulsion according to the invention also advantageously comprises one or more lipid components, such as waxes and oils based on hydrocarbons, saturated, unsaturated or hydrogenated triglycerides, dialkyl ethers having 12 to 24 carbon atoms and/or the esters of monohydric alcohols and monocarboxylic acids having at least 10 carbon atoms.

As already stated, it is advantageous if the O/W emulsion of the present invention does not comprise any mineral oil.

It is also advantageous if no branched and/or unbranched hydrocarbons are present. Examples of such hydrocarbons are paraffinum liquidum, isododecane, isohexadecane, isoeicosane, squalane and cera microcristallina.

The O/W emulsion according to the invention advantageously comprises one or more triglycerol esters selected from the group of synthetic, semi-synthetic and natural triglycerol esters. It is particularly preferred if the oil phase comprises exclusively natural triglycerol esters among the triglycerol esters.

Natural oils advantageously present are selected from the group Persea gratissima oil, Orbignya oleifera seed oil, Argania spinosa kernel oil, Prunus armeniaca kernel oil, Simmondsia chinensis seed oil, Cocos nucifera oil, Silybum marianum seed oil, Oenothera biennis oil, Olea europaea fruit oil, Helianthus annuus seed oil, Vitis vinifera seed oil, Cannabis sativa seed oil, Olus oil, Gossypium herbaceum seed oil, Arctium lappa seed oil, Macadamia ternifolia seed oil, Zea mays germ oil, Prunus amygdalus dulcis oil, Ricinus communis seed oil, vegetable oil and glycine soja oil.

If the O/W emulsion comprises one or more of the natural oils specified above, the fraction of these natural oils is preferably from 0.1% by weight to 5.0% by weight, based on the total weight of the O/W Emulsion.

It is also advantageous if an oil is present selected from the group of coco-caprylate, dicaprylyl ether, cocoglycerides, coco-caprylate/caprate, decyl oleate, caprylic/capric triglyceride, ethylhexyl cocoate, octyldodecanol, squalane, triisostearin, Shea butter ethyl esters, ethylhexyl cocoate, decyl cocoate, isoamyl cocoate, caprylyl caprylate/caprate, triheptanoin, hexyldecyl stearate and isoamyl laurate.

Other preferred triglycerol esters include hydrogenated triglyceride fats such as hydrogenated palm oil, hydrogenated coconut oil, hydrogenated castor oil or tristearin. In particular, the use of hydrogenated coconut oil (hydrogenated coco-glycerides) is preferred, the fraction of hydrogenated coconut oil being preferably from 0.5% by weight to 3% by weight, based on the total weight of the emulsion.

It is also advantageous in the context of the present invention if the O/W emulsion of the present invention comprises one or more dialkyl ethers having 12 to 24 carbon atoms, in which dicaprylyl ether is preferably present. If the O/W emulsion comprises dicaprylyl ether, the fraction of dicaprylyl ether is preferably from 0.1% by weight to 3% by weight, based on the total weight of the O/W emulsion.

It is also advantageous in the context of the present invention if the O/W emulsion comprises cetyl palmitate. If cetyl palmitate is present, the fraction of cetyl palmitate is preferably from 0.1 to 5% by weight, based on the total weight of the emulsion.

It is also advantageous in the context of the present invention if the O/W emulsion comprises octyldodecanol. If octyldodecanol is present, the fraction of octyldodecanol is preferably from 0.1 to 2% by weight, based on the total weight of the emulsion.

Furthermore, it is particularly advantageous if the emulsion is characterized in that it comprises coco-caprylate and/or coco-caprylate/caprate, the total fraction of coco-caprylate and/or coco-caprylate/caprate preferably being from 0.5 to 7% by weight and particularly preferably from 2 to 6% by weight, based on the total weight of the emulsion.

Furthermore, it is particularly advantageous if the emulsion is characterized in that it comprises triisostearin and/or tristearin, the total fraction of triisostearin and/or tristearin preferably being from 0.5 to 7% by weight and particularly preferably from 1.3 to 3.5% by weight, based on the total weight of the emulsion.

Another particularly advantageous constituent of the emulsion is also Butyrospermum parkii butter, which is preferably present at a fraction from 0.3 to 2% by weight and particularly preferably from 0.5 to 1.5% by weight, based on the total weight of the emulsion.

It is also advantageous according to the invention if the O/W emulsion comprises propylene glycol, butylene glycol, 2-methylpropane-1,3-diol, pentane-1,2-diol, hexane-1,2-diol, octane-1,2-diol, glyceryl caprylate and/or decane-1,2-diol.

Furthermore, it is also advantageous in the context of the present invention if the O/W emulsion comprises phenoxyethanol, dehydroacetic acid, benzyl alcohol and/or ethylhexylglycerin.

If the O/W emulsion comprises benzyl alcohol, it is preferred if the fraction of benzyl alcohol is from 0.1% by weight to 0.5% by weight, based on the total weight of the O/W emulsion.

If the O/W emulsion comprises phenoxyethanol, it is preferred if the total fraction of phenoxyethanol is from 0.1% by weight to 1.2% by weight, based on the total weight of the O/W emulsion.

If the O/W emulsion comprises ethylhexylglycerin, it is preferred if the fraction of ethylhexylglycerin is from 0.1% by weight to 1.0% by weight, based on the total weight of the O/W emulsion.

If the O/W emulsion comprises octane-1,2-diol, it is preferred if the fraction of octane-1,2-diol is from 0.1% by weight to 1.0% by weight, based on the total weight of the O/W emulsion.

In addition, it is advantageous when embodiments of the invention are characterized in that these comprise ethanol. If ethanol is present in the O/W emulsion, the fraction of ethanol is preferably from 0.5% by weight to 10% by weight, based on the total weight of the O/W emulsion.

Furthermore, the O/W emulsion according to the invention is preferably characterized in that it comprises glycerol at a fraction of 0.5% by weight to 15% by weight, based on the total weight of the O/W emulsion.

Furthermore, it is preferred if the O/W emulsion comprises further surfactants and/or emulsifiers only at a maximum fraction of 2% by weight, preferably at a maximum fraction of 1.5% by weight, the figures being based on the total weight of the emulsion.

Last but not least, advantageous embodiments according to the invention are characterized in that the O/W emulsion comprises one or more active ingredients selected from the group of the compounds glycyrrhetinic acid, urea, arctiin, folic acid, coenzyme Q10 (ubiquinone), alpha-glucosylrutin, carnitine, carnosine, caffeine, natural and/or synthetic isoflavonoids, glyceryl glucose, creatine, creatinine, taurine, tocopherol, tocopherol acetate, vitamin C, vitamin C phosphate, vitamin C palmitate, niacinamide, vitamin A palmitate, panthenol, licochalcone A, rucinol, N-[(2,4-dihydroxyphenyl)thiazol-2-yl]isobutyramide, honokiol and magnolol (also as a constituent of magnolia extracts), hyaluronic acid and/or silymarin (milk thistle extract).

Furthermore, advantageous O/W emulsions according to the invention are characterized in that said emulsions comprise water at a proportion of 60% by weight to 95% by weight and preferably from 70% by weight to 90% by weight, based on the total weight of emulsion.

Advantageous O/W emulsions according to the invention also have a viscosity from 8000 mPa·s to 20 000 mPa·s 24 h after preparation. If viscosity is referred to in this disclosure, all values relate to a measurement at 25° C. in a 150 ml snap-lid bottle using a Rheomat R 123 from proRheo. The Rheomat R 123 from proRheo GmbH is a rotational viscometer, i.e. a measurement body rotates in the substance to be measured. The force is measured that is required to rotate the measurement body in the sample at a predefined speed. From this torque, the speed of the measurement body and the geometric dimensions of the measuring system used, the viscosity is calculated. The measurement body used is measurement body No. 2 (item no. 200 0192), speed range 62.5 min⁻¹. All viscosity measurements are always carried out 24 hours after preparation of the 0/W emulsion.

### COMPARATIVE EXPERIMENTS AND EXAMPLES

The examples below are intended to illustrate the present invention without limiting it. Unless otherwise stated, all quantitative data, fractions, and percentages are based on the weight and the total amount or on the total weight of the preparations.

Comp. 1 and Comp. 2 in the table below show two emulsions not according to the invention, while Example 1 represents an emulsion according to the invention. The properties of Comp.1 and Comp.2 were compared to Ex.1. When applying the emulsions to the skin, an expert panel found that Comp.1 and Comp.2 left significant amounts of white residues when applied to the forearm. In contrast, Example 1 shows almost no white residues.

| Ingredient | Comp. 1 | Comp. 2 | Ex. 1 |
|---|---|---|---|
| Fatty acid mixture | 3 | 3 | 3 |
| consisting of 47% stearic | | | |
| acid, 48.5% palmitic acid, | | | |
| 2.5% myristic acid, 1.5% | | | |
| arachidic acid and 0.5% | | | |
| oleic acid. | | | |
| Glyceryl stearate | 1.2 | 1.2 | 1.2 |
| Polyglyceryl-3 diisostearate | 0.7 | 0.7 | 0.7 |
| Vegetable oil | 1 | 1 | 1 |
| Octyldodecanol | 0.5 | 0.5 | 0.5 |
| Triisostearin | 1.5 | 1.5 | 1.5 |
| Myristyl alcohol | 5 | 5 | 5 |
| Myristyl myristate | 2 | 2 | 2 |
| Hydrogenated coco-glycerides | 1.2 | 1.2 | 1.2 |
| Phenoxyethanol | 0.35 | 0.35 | 0.35 |
| Ethylhexylglycerin | 0.2 | 0.2 | 0.2 |
| Parfum | 0.1 | 0.1 | 0.1 |
| Sodium hydroxide | q.s. | q.s. | q.s. |
| Hydroxypropyl starch | | | 2 |
| phosphate | | | |
| Gellan gum | 0.1 | | |
| Kappa carrageenan | | 0.4 | |
| Glycerol | 3 | 3 | 3 |
| Ethanol | 3 | 3 | 3 |
| Aqua | to 100 | to 100 | to 100 |
| Whitening when spread on | Strong | Strong | Slight |
| forearm (each 75 μl) | | | |

The further examples are intended to further illustrate the invention without restricting it.

| Ingredient | Ex. 2 | Ex. 3 | Ex. 4 | Ex. 5 | Ex. 6 | Ex. 7 |
|---|---|---|---|---|---|---|
| Fatty acid mixture | 1.5 | 4 | 1 | 2 | 0.8 | 0.5 |
| consisting of 47% stearic | | | | | | |
| acid, 48.5% palmitic acid, | | | | | | |
| 2.5% myristic acid, 1.5% | | | | | | |
| arachidic acid and 0.5% | | | | | | |
| oleic acid. | | | | | | |
| Glyceryl stearate | 1.5 | | | 2 | 1 | 1.2 |
| Glyceryl stearate citrate | 1.5 | 2 | 1 | | 0.8 | 0.6 |
| Polyglyceryl-3 | 0.4 | | 3 | | 0.5 | 0.2 |
| diisostearate + aqua | | | | | | |
| Vegetable oil | 0.1 | | | | 0.6 | 0.8 |
| Prunus amygdalus dulcis | 0.2 | 1 | | | | 0.3 |
| oil | | | | | | |
| Octyldodecanol | | | 0.2 | 1 | 0.4 | |
| Coco-caprylate | | 1 | 0.2 | | | |
| Coco-caprylate/caprate | 0.2 | | | 1 | | |
| Caprylic/capric | 0.2 | | 0.5 | | | |
| triglyceride | | | | | | |
| Dicaprylyl ether | | 0.2 | | | | |
| Triisostearin | | | | 0.2 | 1.2 | |
| Tristearin | | | 0.5 | 0.2 | | 1.2 |
| Myristyl alcohol | | 6 | | | 2 | 3 |
| Cetyl alcohol | | | 3 | | 2 | |
| Cetearyl alcohol | 3 | | | 4 | | |
| Myristyl myristate | 1.5 | 1 | | 0.5 | 1.8 | 3 |
| Hydrogenated coco- | | | | 2 | 1 | 0.1 |
| glycerides | | | | | | |
| Cetyl palmitate | 1 | 2 | 1.5 | | | |
| Butyrospermum parkii | 1 | | 1.5 | 5 | | |
| butter | | | | | | |
| Phenoxyethanol | 0.9 | 0.9 | 0.9 | 0.9 | | |
| Caprylyl glycol | | | | 0.1 | 0.25 | 0.33 |
| Ethylhexyglycerin | | | 0.1 | | 0.25 | 0.4 |
| Polyglyceryl-2 caprate | | 0.5 | | | | |
| Parfum | 0.2 | 0.4 | 0.25 | 0.4 | 0.2 | 0.15 |
| Sodium hydroxide | q.s. | q.s. | q.s. | q.s. | q.s. | q.s. |
| Hydroxypropyl starch | 0.75 | 0.2 | 3 | 3 | 1.5 | 0.8 |
| phosphate | | | | | | |
| Glycerol | 5 | 8 | 7 | 12 | 4 | 5 |
| Ethanol | 4 | 2 | 2 | 4 | 4 | 3 |
| Aqua | to 100 | to 100 | to 100 | to 100 | to 100 | to 100 |

| Ingredient | Ex. 8 | Ex. 9 | Ex. 10 |
|---|---|---|---|
| Fatty acid mixture consisting of 47% | 0.2 | 2 | 1 |
| stearic acid, 48.5% palmitic acid, | | | |
| 2.5% myristic acid, 1.5% arachidic | | | |
| acid and 0.5% oleic acid. | | | |
| Glyceryl stearate | 3 | 0.5 | 0.5 |
| Glyceryl stearate citrate | 2.5 | | 1 |
| Polyglyceryl-3 diisostearate + aqua | | 1 | 0.8 |
| Vegetable oil | | | 0.4 |
| Prunus amygdalus dulcis oil | 0.7 | | 0.5 |
| Coco-caprylate | | 0.5 | |
| Coco-caprylate/caprate | 0.5 | | |
| Caprylic/capric triglyceride | | 1 | |
| Dicaprylyl ether | | 1 | |
| Triisostearin | | 2 | |
| Tristearin | 2.5 | | 2 |
| Myristyl alcohol | | | |
| Cetyl alcohol | | 4 | 3.5 |
| Cetearyl alcohol | 3.5 | | |
| Myristyl myristate | 0.2 | | 2.5 |
| Hydrogenated coco glycerides | | 0.5 | 0.5 |
| Cetyl palmitate | 3 | 0.5 | |
| Butyrospermum parkii butter | | 1 | |
| Caprylyl glycol | 0.33 | 0.25 | 0.1 |
| Ethylhexyglycerin | 0.6 | 0.25 | 0.4 |
| Polyglyceryl-2 caprate | | 0.25 | 0.2 |
| Parfum | 0.4 | 0.25 | 0.3 |
| Aqua + Sodium hydroxide | 0.1 | 0.02 | 0.05 |
| Hydroxypropyl starch phosphate | 2 | 1 | 2.5 |
| Glycerol | 6 | 10 | 6 |
| Ethanol | 4.5 | 2 | 5 |
| Aqua | to 100 | to 100 | to 100 |

1.-15. (canceled)

16. A cosmetic oil-in-water (O/W) emulsion, wherein the emulsion is acrylate- and silicone-free and comprises, based on a total weight of the emulsion,
(a) from 0.1% to 6% by weight of at least one fatty acid having 12 to 22 carbon atoms, and
(b) from 0.1% to 5% by weight hydroxypropyl starch phosphate.

17. The emulsion of claim 16, wherein (a) comprises one or more of palmitic acid, stearic acid, myristic acid, arachidic acid, and oleic acid.

18. The emulsion of claim 16, wherein the emulsion comprises from 0.5% to 3% by weight of (a).

19. The emulsion of claim 16, wherein (a) comprises palmitic acid.

20. The emulsion of claim 19, wherein the emulsion comprises from 0.4% to 2.2% by weight of palmitic acid.

21. The emulsion of claim 16, wherein (a) comprises stearic acid.

22. The emulsion of claim 21, wherein the emulsion comprises from 0.4% to 2.2% by weight of stearic acid.

23. The emulsion of claim 16, wherein the emulsion comprises from 0.2% to 3.8% by weight of (b).

24. The emulsion of claim 16, wherein the emulsion comprises from 0.4% to 3% by weight of (b).

25. The emulsion of claim 16, wherein the emulsion further comprises from 0.1% to 5.0% by weight of glyceryl stearate, based on a total weight of the emulsion.

26. The emulsion of claim 16, wherein the emulsion further comprises from 0.1% to 5.0% by weight of glyceryl stearate citrate, based on a total weight of the emulsion.

27. The emulsion of claim 16, wherein the emulsion further comprises polyglyceryl-3 diisostearate.

28. The emulsion of claim 16, wherein the emulsion comprises from more than 2% by weight to 30% by weight of an oil phase, based on a total weight of the emulsion.

29. The emulsion of claim 28, wherein the emulsion comprises from more than 5% by weight to 20% by weight of the oil phase.

30. The emulsion of claim 28, wherein the emulsion comprises from more than 8% by weight to 15.5% by weight of the oil phase.

31. The emulsion of claim 16, wherein the emulsion further comprises one or more fatty alcohols having 14 to 22 carbon atoms.

32. The emulsion of claim 16, wherein the emulsion does not comprise any mineral oil.

33. The emulsion of claim 16, wherein the emulsion does not comprise more than 2% by weight of further surfactants and/or emulsifiers, based on a total weight of the emulsion.

34. The emulsion of claim 16, wherein apart from (b), the emulsion does not comprise any other starches and starch derivatives.

35. A method of reducing white residues caused by applying an acrylate- and silicone-free O/W emulsion onto skin, wherein the method comprises including in the emulsion a mixture of
(a) at least one fatty acid having 12 to 22 carbon atoms, and
(b) hydroxypropyl starch phosphate.
